(19) United States
(12) Patent Application Publication
(10) Pub. No.: US 2008/0274073 A1
(43) Pub. Date: Nov. 6, 2008

(54) Process for Producing Aqueous Emulsions and Dispersions

(75) Inventors: Hubertus Peter Bell, Mannheim (DE); Stephan Huffer, Ludwigshafen (DE); Matthias Kluglein, Ludwigshafen (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE Patents, Trademarks and Licenses, Ludwigshafen (DE)

(21) Appl. No.: 12/090,160

(22) PCT Filed: Oct. 5, 2006
(86) PCT No.: PCT/EP2006/067072

§ 371 Date: Apr. 14, 2008

(30) Foreign Application Priority Data
Oct. 12, 2005 (DE) 102005049327.0

(51) Int. Cl. A61K 8/81; C08F 222/06; A61Q 99/00; C10M 169/04

(52) U.S. Cl. 424/78.02; 526/272; 508/306

(57) ABSTRACT

The use of terpolymers, obtainable by free radical copolymerization of
(a) at least one anhydride of a C₃-C₁₀-dicarboxylic acid,
(b) at least one 1,1-di-(C₁-C₃-alkyl)-substituted C₄-C₈-olefin,
(c) polyisobutene having an average molecular weight Mₙ in the range of from 200 to 10 000 g/mol
   and, if appropriate hydrolysis,
   for the preparation of aqueous emulsions or dispersions of hydrophobic substances with the use of not more than 2% by weight of further emulsifier, based on the total aqueous emulsion or dispersion.

The present invention relates to the use of terpolymers (A) obtainable by free radical copolymerization of
(a) at least one anhydride of a C₃-C₁₀-dicarboxylic acid,
(b) at least one 1,1-di-(C₁-C₃-alkyl)-substituted C₄-C₈-olefin,
(c) polyisobutene having an average molecular weight Mₙ in the range of from 200 to 10 000 g/mol
   and, if appropriate, hydrolysis,
   for the preparation of aqueous emulsions or dispersions of silicones (B) with the use of not more than 2% by weight of further emulsifier, based on the total aqueous emulsion or dispersion.

The present invention furthermore relates to a process for the preparation of aqueous emulsions or dispersions of silicones (B) with the use of terpolymers (A) obtainable by free radical copolymerization of
(a) at least one anhydride of C₃-C₁₀-dicarboxylic acid,
(b) at least one 1,1-di-(C₁-C₃-alkyl)-substituted C₄-C₈-olefin,
(c) polyisobutene having an average molecular weight Mₙ in the range of from 200 to 10 000 g/mol
   and, if appropriate, hydrolysis
   and with the use of not more than 2% by weight of further emulsifier, based on the total aqueous emulsion or dispersion.

Silicones are substances which can be widely used. In many cases, it is therefore desired to apply silicones. The imparting of water repellency to and greasing, lubricating or impregnating of surfaces and furthermore the imparting of water repellency to and greasing of fibrous substrates, in particular of water-swellable substrates, such as, for example, of tanned animal hides, may be mentioned by way of example. Owing to the circumstances, however, it is intended to carry out the application in the aqueous phase and not with the use of organic solvents, since organic solvents may, for example, be very flammable or have physiologically disadvantageous properties. It is therefore necessary to disperse or emulsify the hydrophobic substances to be applied. Such emulsions should have advantageous properties, which include in particular the stability, i.e. they should not exhibit measurable separation or should separate only within a long time. The preparation of so-called stable emulsions and dispersions of silicones is therefore an important working area.

For the preparation of stable emulsions and dispersions, the choice of the emulsifier therefore plays a key role. Many customary emulsifiers can in the meantime become undesirable because they adversely influence the waste waters and may have, for example, a high COD (chemical oxygen demand) or BOD (biological oxygen demand) which makes the waste water disposal more expensive.

U.S. Pat. No. 3,004,950 proposes special block copolymers of vinyisilicone compounds with, for example, N-vinylpyrrolidone as emulsifiers. U.S. Pat. No. 6,239,290 proposes sorbitan derivatives containing silicone groups as emulsifiers. However, such compounds are in each case inconvenient to synthesize.

It was therefore the object to provide a process by means of which silicones can be emulsified or dispersed in water and which avoids the abovementioned disadvantages.

Accordingly, the use defined at the outset and the process defined at the outset were found.

The present invention relates to the use of terpolymers (A) obtainable by free radical copolymerization of
(a) at least one anhydride of a C₃-C₁₀-dicarboxylic acid,
(b) at least one 1,1-di-(C₁-C₃-alkyl)-substituted C₄-C₈-olefin,
(c) polyisobutene having an average molecular weight Mₙ in the range of from 200 to 10 000 g/mol,
   also referred to as terpolymers (A) for short in the context of the present invention, for the preparation of aqueous emulsions or dispersions of silicones (B) with the use of not more than 2% by weight of further emulsifier, based on the total aqueous emulsion or dispersion.

The present invention furthermore relates to a process for the preparation of aqueous emulsions or dispersions of silicones (B) with the use of terpolymers (A) obtainable by free radical copolymerization of
(a) at least one anhydride of a C₃-C₁₀-dicarboxylic acid,
(b) at least one 1,1-di-(C₁-C₃-alkyl)-substituted C₄-C₈-olefin,
(c) polyisobutene having an average molecular weight Mₙ in the range of from 200 to 10 000 g/mol,
   and with the use of not more than 2% by weight of further emulsifier, based on total aqueous emulsion or dispersion.

In the context of the present invention, silicones (B) are understood as meaning compounds which are solid or preferably liquid at room temperature and are substantially composed of the same or different groups of the formula I

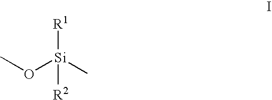

Here, R¹ and R² are different or preferably identical and are selected from phenyl, (CH₂)ₙ—COOH, (CH₂)ₙ—CH(COOH)—CH₂—COOH, n being an integer in the range of from 1 to 30, preferably from 11 to 25, perfluoroalkyl, such as, for example, trifluoromethyl, n-C₃F₇ and n-C₄F₉, and in particular C₁-C₂₀-alkyl, such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, n-nonyl, n-decyl, n-eicosyl; preferably linear C₁-C₄-alkyl, such as methyl, ethyl, n-propyl, n-butyl and very particularly methyl.

If at least one R¹ or R² is selected from (CH₂)ₙ—COOH, preferably not all R¹ and R² are identical.

In an embodiment of the present invention, silicones (B) have an average molecular weight Mₙ in the range of from 500 to 100 000 g/mol, preferably from 2500 to 25 000 g/mol.

Silicones (B) may be cyclic, branched or preferably linear; in the latter case, the free valency is saturated with an R¹, in particular with methyl.

In an embodiment of the present invention, silicones (B) have an average molecular weight Mₙ in the range of from 8000 to 11 000 g/mol.

In an embodiment of the present invention, silicones (B) carry on average one carboxyl group per molecule. In another embodiment of the present invention, silicones (B) carry on average two or three or four carboxyl groups per molecule. In another embodiment of the present invention, silicones (B) carry no carboxyl groups (B).

In an embodiment of the present invention, carboxyl groups of silicones (B) may be neutralized, for example with alkaline metals, such as, for example, potassium or sodium.

According to the invention, one or more terpolymers (A), obtainable by free radical copolymerization of,
(a) at least one anhydride of a C₃-C₁₀-dicarboxylic acid, for example citraconic anhydride, itaconic anhydride and in particular maleic anhydride,
(b) at least one 1,1-di-(C₁-C₃-alkyl)-substituted C₄-C₈-olefin, for example 2-methyl-1-butene, 2-ethyl-1-butene, diisobutene (mixture of 2,4,4-trimethyl-1-pentene and 2,4,4-trimethyl-2-pentene), 2-ethylpent-1-ene and 2-ethylhex-1-ene and in particular isobutene,
(c) polyisobutene having an average molecular weight Mₙ in the range of from 200 to 10 000 g/mol, preferably from 500 to 1000 g/mol, are used for dispersing or preferably emulsifying silicone (B) characterized above. Polyisobutene is one which has one terminal ethylenically unsaturated group per molecule, for example a ylnyl, vinylidene or alkylvinylidene group.

In an embodiment of the present invention, terpolymer (A) has an average molecular weight Mₙ in the range of from 500 to 50 000 g/mol, preferably from 1500 to 20 000 g/mol, determined, for example, by gel permeation chromatography (GPC).

In an embodiment of the present invention, the polydispersity of terpolymer (A) is in the range of from 1.1 to 10, preferably from 1.5 to 3.0.

In an embodiment of the present invention, polyisobutene incorporated into terpolymer (A) and having an average molecular weight Mₙ in the range of from 200 to 10 000 g/mol (c) has a polydispersity in the range of from 1.1 to 3, preferably from 1.5 to 2.0.

Polyisobutene having an average molecular weight Mₙ in the range of from 200 to 10 000 g/mol (c) and its preparation are known as such, cf. for example DE-A 27 02 604.

In an embodiment of the present invention, terpolymer (A) is a quasi-alternating terpolymer, i.e. in the present case two units of anhydride of C₃-C₁₀-dicarboxylic acid (a) are not incorporated directly adjacent into the polymer chain of terpolymer (A) but in each case are interrupted by at least one unit of a 1,1-di-(C₁-C₃-alkyl)-substituted C₄-C₈-olefin (b) or at least one unit of polyisobutene having an average molecular weight Mₙ in the range from 200 to 10 000 g/mol (c), before the next unit of anhydride of C₃-C₁₀-dicarboxylic acid (a) is incorporated into terpolymer (A).

The free radical copolymerization of
(a) at least one anhydride of a C₃-C₁₀-dicarboxylic acid,
(b) at least one 1,1-di-(C₁-C₃-alkyl)-substituted C₄-C₈-olefin,
(c) polyisobutene having an average molecular weight Mₙ in the range of from 200 to 10 000 g/mol
   can be carried out with or without diluents, for example one or more solvents or one of more precipitating agents. Suitable solvents for the free radical copolymerization are polar solvents inert to acid anhydride, such as, for example, acetone, tetrahydrofuran and 1,4-dioxane or toluene, ortho-xylene, meta-xylene and aliphatic hydrocarbons.

The free radical copolymerization of
(a) at least one anhydride of a C₃-C₁₀-dicarboxylic acid,
(b) at least one 1,1-di-(C₁-C₃-alkyl)-substituted C₄-C₈-olefin,
(c) polyisobutene having an average molecular weight Mₙ in the range of from 200 to 10 000 g/mol
   is preferably carried out using an initiator or an initiator system. Suitable initiators are, for example, organic peroxides or hydroperoxides. Di-tert-butyl peroxide, tert-butyl perpivalate, tert-butyl per-2-ethylhexanoate, tert-butyl permaleate, tert-butyl perisobutyrate, benzoyl peroxide, diacetyl peroxide, succinyl peroxide, p-chlorobenzoyl peroxide and dicyclohexyl peroxodicarbonate being mentioned by way of example. The use of initiator systems, such as, for example, redox initiators, is also suitable, for example combinations of hydrogen peroxide or sodium peroxodisulfate or one of the above mentioned peroxides with a reducing agent. Examples of suitable reducing agents are, ascorbic acid, tartaric acid, Fe(II) salts, such as, for example FeSO₄, sodium bisulfite and potassium bisulfite.

Other suitable initiators are azo compounds, such as 2,2′-azobisisobutyronitrile, 2,2′-azobis(2-methylpropionamidine) dihydrochloride and 2,2′-azobis(4-methoxy-2,4-dimethylvaleronitrile).

In general, initiator is used in amounts of from 0.1 to 20% by weight, preferably from 0.2 to 15% by weight, calculated relative to the mass of all comonomers (a) to (c).

After the free radical copolymerization, hydrolysis can be effected, for example, by adding water, which, if appropriate, may comprise one or more Brønsted bases. Examples of Brønsted bases are alkali metal hydroxide, such as, for example, NaOH and KOH, alkali metal carbonate such as, for example, Na₂CO₃ and K₂CO₃, alkali metal bicarbonate, such as, for example, NaHCO₃ and KHCO₃, ammonia, amines, such as, for example, trimethylamine, triethylamine, diethylamine, ethanolamine, N,N-diethanolamine, N,N,N-triethanolamine and N-methylethanolamine.

Terpolymer (A) can be used in unhydrolyzed or preferably hydrolyzed form for carrying out the process according to the invention.

Further emulsifier, of which, according to the invention, not more than 2% by weight, based on the total aqueous emulsion or dispersion, preferably from 0.0001 to 1.5% by weight, can be used, is selected, for example, from ionic, i.e. anionic or cationic, emulsifiers, and preferably nonionic emulsifiers, in particular sodium N-methyl-N-acyltaurates and N-acylsarcosines, and mono- or polyalkoxylated phosphate esters, in particular phosphate esters having a degree of ethoxylation of from 3 to 100, alcohols, sorbitan esters, fatty acids, fatty amines, fatty amides or esters, and polyethylene glycol esters.

Polyalkoxylated C₁₀-C₄₀-fatty alcohols, in particular those having a degree of ethoxylation of up to 100 are preferred, and preferably polyalkoxylated C₁₁-C₃₁-oxo alcohols, in particular those having a degree of ethoxylation of up to 100. Examples of particularly suitable fatty alcohols and polyalkoxylated oxo alcohols are those of the general formula II

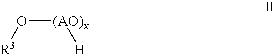

in which the variables are defined as follows:
R³ is branched or straight-chain C₆-C₃₀-alkyl or C₆-C₃₀-alkenyl, preferably C₈-C₂₀-alkyl or C₈-C₂₀-alkenyl, it being possible for C₆-C₃₀-alkenyl to have one or more C—C double bonds which may preferably have the (Z)-configuration,
AO is C₂-C₄-alkylene oxide, identical or different, for example butylene oxide CH(C₂H₅)CH₂O, propylene oxide CH(CH₃)CH₂O and in particular ethylene oxide CH₂CH₂O,
x is a number in the range of from 2 to 100, it being possible for x as an average value (number average) also to be a nonintegral number, preferably in the range of from 2 to 90 and particularly from 2.5 to 80.

Where AO are different alkylene oxides, the different alkylene oxides may be arranged in blocks or randomly.

Examples of particularly suitable polyalkoxylated fatty alcohols and oxo alcohols of
n-C₁₈H₃₇O—(CH₂CH₂O)₈₀—H,
n-C₁₈H₃₇O—(CH₂CH₂O)₇₀—H,
n-C₁H₃₇O—(CH₂CH₂O)₆₀—H,
n-C₁₈H₃₇O—(CH₂CH₂O)₅₀—H,
n-C₁₈H₃₇O—(CH₂CH₂O)₂₅—H,
n-C₁₈H₃₇O—(CH₂CH₂O)₁₂—H,
n-C₁₆H₃₃O—(CH₂CH₂O)₅₀—H,
n-C₁₆H₃₃O—(CH₂CH₂O)₇₀—H,
n-C₁₆H₃₃O—(CH₂CH₂O)₆₀—H,
n-C₁₆H₃₃O—(CH₂CH₂O)₅₀—H,
n-C₁₆H₃₃O—(CH₂CH₂O)₂₅—H,
n-C₁₆H₃₃O—(CH₂CH₂O)₁₂—H,
n-C₁₂H₂₅O—(CH₂CH₂O)₁₁—H,
n-C₁₂H₂₅O—(CH₂CH₂O)₁₈—H,
n-C₁₂H₂₅O—(CH₂CH₂O)₂₅—H,
n-C₁₂H₂₅O—(CH₂CH₂O)₅₀—H,
n-C₁₂H₂₅O—(CH₂CH₂O)₈—H,
n-C₃₀H₆₁O—(CH₂CH₂O)₈—H,
n-C₁₀H₂₁O—(CH₂CH₂O)₉—H,
n-C₁₀H₂₁O—(CH₂CH₂O)₇—H,
n-C₁₀H₂₁O—(CH₂CH₂O)₅—H,
n-C₁₀H₂₁O—(CH₂CH₂O)₃—H,
   and mixtures of the abovementioned emulsifiers, for example mixtures of n-C₁₈H₃₇O—(CH₂CH₂O)₅₀—H and n-C₁₆H₃₃O—(CH₂CH₂O)₅₀—H,
   where the indices in each case are to be regarded as average values (number average).

An example of a particularly suitable N-acylsarcosine is N-oleoyl-N-sarcosine.

In a special embodiment of the present invention no further emulsifier is used for emulsifying or dispersing silicone (B).

In a preferred embodiment of the present invention, no anionic emulsifier is used for emulsifying a dispersing silicone (B).

In an embodiment of the present invention, the process according to the invention is carried out by a procedure in which two or more further emulsifiers are used for emulsifying or dispersing silicone (B), it being ensured that the sum of further emulsifiers does not exceed 2% by weight, based on the total aqueous emulsion or dispersion.

In an embodiment of the present invention, aqueous emulsions or dispersions prepared according to the invention have a water content in the range of from 40 to 95% by weight, preferably from 60 to 90% by weight.

In an embodiment of the present invention the mixing is carried out at temperatures in the range of from 0 to 100° C., preferably in the range of from 20 to 50° C.

The mixing can be carried out at any desired pressure, atmospheric pressure being preferred.

In an embodiment of the present invention the process according to the invention is carried out by a procedure in which terpolymer (A), silicone (B) and not more than 2% by weight of emulsifier are mixed, for example shaken or preferably stirred, with water.

In an embodiment of the present invention the process according to the invention is carried out by a procedure in which terpolymer (A), silicone (B) and not more than 2% by weight of emulsifier are mixed, in particular homogenized, by application of ultrasound or by means of a gap homogenizer.

In an embodiment of the present invention the process according to the invention is carried out by a procedure in which terpolymer (A), silicone (B) and from zero to not more than 2% by weight of emulsifier are mixed with water and one or more organic solvent selected from aliphatic and aromatic hydrocarbons which are liquid at room temperature, and organic solvent is then separated off. Aliphatic solvents which are liquid at room temperature may be selected, for example, from cyclohexane, cycloheptane, n-hexane, n-heptane, isododecane, n-decane, n-octane and isooctane. Aromatic solvents which are liquid at room temperature may be selected, for example, from benzene, preferably toluene, ethylbenzene, cumene, ortho-xylene, meta-xylene, para-xylene and isomer mixtures of xylene. Organic solvent can be separated off, for example, by distillation, in particular by steam distillation.

In an embodiment of the present invention, from 10 to 40% by weight of organic solvents are used based on water.

In an embodiment of the present invention, mixing can be effected with at least one further hydrophobic substance (C). Suitable further hydrophobic substances are, for example, polyolefins, in particular polyisobutene, for example having a molecular weight Mₙ in the range of from 500 to 20 000 g/mol.

In an embodiment of the present invention, aqueous emulsions or dispersions prepared according to the invention comprise

in the range of from 1 to 60% by weight, preferably from 2 to 20% by weight, of terpolymer (A),
in the range of from 1 to 60% by weight, preferably from 2 to 40% by weight, of silicone (B),
altogether in the range of from 0 to 50% by weight, preferably from 2 to 30% by weight, of further hydrophobic substance (C),
from 0 to not more than 2% by weight of further emulsifier,
the remainder preferably water.

The present invention furthermore relates to aqueous dispersions or emulsions prepared by the abovedescribed process according to the invention. Aqueous dispersions and emulsions according to the invention are distinguished, for example, by very good stability. Furthermore, with the use of dispersions or emulsions according to the invention, silicone (B) is thoroughly consumed. Wastes of dispersions or emulsions according to the invention can therefore be readily disposed of, and the chemical or biological oxygen demand is low.

Aqueous dispersions or emulsions according to the invention preferably comprise no organic solvent. In the context of the present invention, this is to be understood as meaning that the content of organic solvent, such as, for example, aliphatic or aromatic organic solvent, is less than 0.1% by weight, preferably less than 0.01% by weight, based on aqueous dispersion or emulsion according to the invention.

In an embodiment of the present invention dispersions or emulsions according to the invention have a pH in the range of from 4 to 10, preferably from 6 to 7.

Furthermore, one or more biocides may be added to the emulsions or dispersions according to the invention. For example, isothiazolinones, for example BIT: 1,2-benzoisothiazol-3(2H)-one, CIT:5-chloro-2-methyl-2H-isothiazol-3-one; and MIT:2-methyl-2H-isothiazol-3-one, parabens, e.g. methylparaben, ethylparaben or propylparaben.

In an embodiment of the present invention, aqueous emulsions or dispersions according to the invention comprise

In the range of from 1 to 60% by weight, preferably from 2 to 20% by weight of terpolymer (A),
In the range of from 1 to 60% by weight, preferably from 2 to 40% by weight, of silicone (B),
altogether in the range of from 0 to 50% by weight, preferably from 2 to 30% by weight, of further hydrophobic substance (C),
from 0 to not more than 2% by weight of, further emulsifier,
the remainder preferably being water.

The present invention furthermore relates to the use of dispersions or emulsions according to the invention for the production of fibrous substrates, as release agents, as lubricants, as cleaning agents, for the treatment or processing of construction materials or as or in cosmetic preparations.

The present invention furthermore relates to processes for the production of fibrous substrates and methods for the cleaning of surfaces, for the separation of articles, for the treatment or processing of construction materials or as or in cosmetic preparations with the use of dispersions or emulsions according to the invention.

Fibrous substrates can be selected, for example, from paper, wood, textile, board and preferably leather.

The present invention furthermore relates to a method for reducing friction between moving parts, for example comprising metal, with the use of dispersions or emulsions according to the invention, for example as lubricants. By means of the method according to the invention for reducing the friction between moving parts, for example comprising metal, the tendency of said parts to undergo corrosion is also reduced and the life is increased.

If it is desired to use emulsions or dispersions according to the invention for the production of fibrous substrates, it is possible, for example, to adopt a procedure in which leather, paper, wood, board or textile is brought into contact, for example, coated, sprayed or impregnated, with emulsion or dispersion according to the invention as such or in a form diluted with water, and then allowed to dry.

If it is desired to use emulsions or dispersions according to the invention for the production of leather, it is preferable to use one or more emulsions or dispersions according to the invention, for example in the tanning or preferably in the retanning or hydrophobing. Such a process according to the invention for tanning, retanning or leather hydrophobing is also referred to below as tanning process according to the invention, retanning process according to the invention and leather hydrophobing process according to the invention, respectively.

The tanning process according to the invention is carried out in general in a manner such that emulsion or dispersion according to the invention is added in one portion or in a plurality of portions immediately before or during the tanning. The tanning process according to the invention is preferably carried out at a pH from 2.5 to 4, it frequently being observed that the pH increases by about 0.3 to three units while the tanning process according to the invention is being carried out. The pH can also be increased by about 0.3 to three units by adding basifying agents.

The tanning process according to the invention is generally carried out at temperatures of from 10 to 45° C., preferably at from 20 to 30° C. A duration of from 10 minutes to 12 hours has proven useful, from one to three hours being preferred. The tanning process according to the invention can be carried out in any desired vessels customary in tanning, for example by drumming in barrels or in rotated drums.

In one variant of the tanning process according to the invention, emulsion or dispersion according to the invention is used together with one or more conventional tanning agents, for example, with chrome tanning agents, or mineral tanning agents, preferably with syntans, polymer tanning agents or vegetable tanning agents, as described, for example, in Ulmann's Encyclopedia of Industrial Chemistry, volume A15, pages 259 to 282 and in particular page 268 et seq., 5^{th} edition (1990) Verlag Chemie Weiheim.

In an embodiment of the tanning process according to the invention emulsion or dispersion according to the invention can be used together with one or more fatliquoring agents and water repellents.

In another embodiment of the tanning process according to the invention the use of fatliquiring agents and water repellants is dispensed with.

The process according to the invention for the treatment of leather can preferably be carried out as a process for retanning leather with the use of emulsion or dispersion according to the invention. The retanning process according to the invention starts from conventionally tanned semi finished products, i.e. semi finished products tanned, for example, with chrome tanning agents or mineral tanning agents, preferably with polymer tanning agents, aldehydes, syntans or resin tanning agents. According to the invention, for carrying out the retanning process according to the invention, emulsion or dispersion according to the invention, as such or preferably in a form diluted with water, is allowed to act on semi finished products.

The retanning process according to the invention can be carried out under conditions otherwise customary in tanning. Expediently, one or more, i.e. from 2 to 6, soaking steps are chosen and washing with water can be effected between the soaking steps. The temperature during the individual soaking steps is in each case in the range of from 5 to 60° C., preferably from 20 to 45° C.

In one embodiment of the retanning process according to the invention, further fatliquoring agents and water repellants can be used.

In another embodiment of the retanning process according to the invention, the use of further fatliquoring agents and water repellants is dispensed with. In the range of from 0.5 to 10% by weight of emulsion or dispersion according to the invention can be metered, % by weight being based on the shaved weight of the leather treated according to the invention or of the semifinished products treated according to the invention.

For carrying out the tanning process or retanning process according to the invention, agents usually used, for example fatliquors, polymer tanning agents, acrylate- and/or methacrylate-based or silicone-based fatliquoring agents, retanning agents based on resin and vegetable tanning agents, fillers or leather dyes or combinations of at least two of the abovementioned substances, can of course be added during the tanning or retanning.

In one embodiment of the present invention, from 0.01 to 10% by weight of dispersion or emulsion according to the invention, based on the shaved weight, are used.

If it is desired to use emulsions or dispersions according to the invention as cleaning agent or for the cleaning of surfaces, it is possible to start from any desired surfaces, for example leather, plastic or other. For example, it is possible to adopt a procedure in which emulsion or dispersion according to the invention is applied, for example using a cleaning means, such as, for example, wadding, a sponge, paper cloth, rag or cloth, or by means of a spray apparatus, for example a spray can, and then allowed to act, for example, from 10 seconds to one day and supernatant emulsion or dispersion according to the invention is then removed, for example using a cleaning means, such as, for example, wadding, a sponge, paper cloth, rag or cloth. Shiny, cleaned surfaces are obtained.

If it is desired to use emulsion or dispersion according to the invention for treating or processing construction materials, the hydrophobing of gypsum, stone, clinker and concrete is preferred. For this purpose, dispersion or emulsion according to the invention is either introduced into concrete raw material or applied subsequently to the relevant construction material, for example by coating, spraying or impregnation, and then allowed to dry.

The present invention furthermore relates to construction materials produced using at least one dispersion or emulsion according to the invention.

If it is desired to use an emulsion or dispersion according to the invention as or in cosmetic preparations, ointments, creams, soaps, lotions, shampoos and hair-care compositions and washing, shower and bath preparations are preferred. The present invention furthermore relates to cosmetic preparations prepared with the use of at least one emulsion or dispersion according to the invention. Cosmetic preparations according to the invention comprise emulsion or dispersion according to the invention.

Cosmetic preparations according to the invention may comprise an oil or fat phase (D) in addition to water and dispersion or emulsion according to the invention. The oil or fat phase (D) may be formed, for example, by one or more natural or synthetic oils, fats or waxes.

In one embodiment of the present invention the oil or fat phase (D) is composed of one or preferably more constituents which are mentioned below.

Constituents of the oil and/or fat phase (D) can be selected, for example, from the group consisting of the lecithins and the fatty acid triglycerides, for example the triglyceryl esters of saturated and/or unsaturated, branched and/or straight-chain alkane carboxylic acids having a chain length of 8 to 24, in particular 12 to 18, carbon atoms. Fatty acid triglycerides can advantageously be selected, for example, from the group consisting of the synthetic, semisynthetic and natural oils, such as, for example, olive oil, sunflower oil, soybean oil, peanut oil, rapeseed oil, almond oil, palm oil, coconut oil, castor oil, wheatgerm oil, grapeseed oil, safflower oil, evening primrose oil and macadamia nut oil.

Further constituent of the oil and/or fat phase (D) can be selected from the group consisting of the esters of saturated and/or unsaturated, branched and/or straight-chain alkane carboxylic acids having a chain length of 3 to 30 carbon atoms and saturated and/or unsaturated, branched and/or straight-chain alcohols having a chain length of 3 to 30 carbon atoms and from the group consisting of the esters of aromatic carboxylic acids and saturated and/or unsaturated, branched and/or straight-chain alcohols having a chain length of 3 to 30 carbon atoms. Preferred examples are isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyledodecyl palmitate, oleyl oleate, oleyl erucate, erucyl oleate, erucyl erucate, dicaprylyl carbonate (cetiol CC) and cocoglycerides (for example Myritol 331), butylene glycol dicaprylate/dicaprate and di-n-butyl adipate, and synthetic, semisynthetic and natural mixtures of such esters, such as, for example, jojoba oil.

Further constituents of the oil or fat phase (D) can be selected from the group consisting of the branched and straight-chain hydrocarbons and hydrocarbon waxes and of the di-C₅-C₂₀-alkylethers, and from the group consisting of the saturated or unsaturated, branched or straight-chain C₁₂-C₃₀-alcohols, which can also perform a foam former function.

Any desired mixtures of abovementioned constituents can also be used as the oil or fat phase (D) in cosmetic preparations according to the invention.

It may be advantageous to use waxes, for example, cetyl palmitate, as the sole lipid component of the oil phase.

Preferred constituents of the oil or fat phase (D) are selected from the group consisting of 2-ethylhexyl isostearate, octyidodecanol, isotridecyl isononanoate, isoeicosane, 2-ethylhexyl cocoate, C₁₂-C₁₅-alkyl benzoate, caprylic/capric acid triglyceride and dicaprylyl ether.

Examples of preferred mixtures of constituents of the oil or fat phase (D) are selected from mixtures of C₁₂-C₁₅-alkyl benzoate and 2-ethylhexyl isostearate, mixtures of C₁₂-C₁₅-alkyl benzoate and isotridecyl isononanoate and mixtures of C₁₂-C₁₅-alkyl benzoate, 2-ethylhexyl isostearate and/or isotridecyl isononanoate.

Fatty acid triglycerides, in particular soybean oil and/or almond oil, are particularly preferably used according to the invention as oils having a polarity of from 5 to 50 mN/m.

From the group consisting of the hydrocarbons, for example, liquid paraffin, squalane, squalene and in particular optionally hydrogenated polyisobutenes may be used as oil or fat phase (D).

In one embodiment of the present invention, the oil or fat phase (D) can be selected from Guerbet alcohols. Guerbet alcohols as such are known and are obtainable, for example, by heating two equivalents of alcohol of the general formula R⁴—CH₂—CH₂—OH in the presence of, for example, Na and/or Cu to give alcohols of the formula R⁴—CH₂—CH₂—CHR⁴—CH₂—OH. Here, R⁴ is C₂-C₂₀-alkyl, branched or preferably straight-chain, in particular straight-chain C₃-C₁₄-alkyl, for example in each case straight-chain propyl, butyl, pentyl, hexyl, heptyl, octyl, nonyl, decyl, undecyl, dodecyl, tridecyl or tetradecyl. Guerbet alcohols which are particularly preferably suitable as oil or fat phase (D) are 2-n-butyloctanol (R⁴=n-C₄H₉) and 2-n-hexyldecanol (R⁴=n-C₆H₁₃) and mixtures of the abovementioned Guerbet alcohols.

Cosmetic preparations according to the invention may furthermore comprise one or more fragrances or aromas (E).

Suitable fragrances or aromas (E) may be pure substances or mixtures of natural or synthetic volatile compounds which produce an odor. Natural fragrances are extracts of flowers (lilly, lavender, rose, jasmine, neroli, ylang ylang), stems and leaves (geranium, patchouli, petitgrain), fruits (aniseed, coriander, cumin, juniper), peels (bergamot, lemon, orange), roots (mace, angelica, celery, cardamom, costus, iris, calmus), woods (pinewood, sandalwood, guajac wood, cedar wood and rosewood) herbs and grasses (tarragon, lemongrass, sage, thyme), needles and twigs (spruce, fir, pine, dwarf pines), resins and balsams (galbanum, elemi, benzoin, myrrh, olibanum, opoponax). Furthermore animal raw materials are suitable, such as, for example, civet and castoreum. Typical synthetic fragrances are products of the ester, ether, aldehyde, ketone, alcohol and hydrocarbon type. Fragrances of the ester type are, for example, benzyl acetate, phenoxyethyl isobutyrate, 4-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The fragrances of the ether type include, for example, benzyl ethyl ether, the fragrances of the aldehyde type include, for example, linear alkanals having 8 to 18 carbon atoms, citral, citronellal, citronellyloxyacetaldehyde, cyclamen aldehyde, hydroxycitronellal, lilial and bourgeonal, the fragrances of the ketone type include, for example, ionones, cc-isomethylionene and methyl cedryl ketone, the fragrances of the alcohol type include anethol, citronellol, eugenol, isoeugenol, geraniol, linalool, benzyl alcohol, phenylethyl alcohol and terioneol, the fragrances of the hydrocarbon type include mainly the terpenes and balsams. However, mixtures of different fragrances which together produce an appealing note are preferably used. Essential oils of low volatility, which are generally used as aroma components, are also suitable as fragrances, e.g. sage oil, cammomile oil, clove oil, melissa oil, mint oil, cinnamon leaf oil, lime flower oil, juniper berry oil, vetiver oil, olibanum oil, galbanum oil, labolanum oil and lavender oil. In bergamot oil, dihydromyrcenol, lilial, lyral, citronellol, phenylether alcohol, α-hexylcinnamaldehyde, α-amylcinnamaldehyde, geraniol, benzylacetone, cyclamen aldehyde, linalool, Boisambrene®Forte, ambroxan, indol, hedione, sandelice, citrus oil, mandarin oil, orange oil, allylamyl glycolate, cyclovertal, lavender oil, Muscat oil, sage oil, β-damascone, Bourbon geranium oil, cyclohexyl salicylate, evernyl, iraldein gamma, phenyl acetic acid, geranyl acetate, benzyl acetate, rose oxide, romillate, irotyl and floramate are preferably used, alone or as mixtures.

Cosmetic preparations according to the invention may furthermore comprise one or more additives (F). Additives (F) can be selected from conditioning agents, antioxidants, ethoxylated glyceryl mono- or di-fatty acid esters, thickeners, foam formers, wetting agents and humectants, biocides, organic solvents, such as, for example, ethanol or isopropanol, glitter and/or other effect substances (e.g. color streaks) and abrasives. Glitter and other effect substances (e.g. color streaks) are substantially of aesthetic importance.

Examples of conditioners are mentioned in International Cosmetic Ingredient Dictionary and Handbook (volume 4, editors: R. C. Pepe, J. A. Wenninger, G. N. McEwen, The Cosmetic, Toiletry, and Fragrance Association, 9^{th} edition 2002) under Section 4 under the keywords Hair Conditioning Agents, Humectants, Skin-Conditioning Agents, SkinConditioning Agents-Emollient, Skin-Conditioning Agents-Humectant, SkinConditioning Agents-Miscellaneous, Skin-Conditioning Agents-Occlusive and Skin Protectants. Further examples of conditioners are compounds mentioned in EP-A 0 934 956 (pages 11 to 13) under “water soluble conditioning agent” and “oil soluble conditioning agent”. Further advantageous conditioners are, for example, the compounds designated as polyquaternium according to INCI (in particular polyquaternium-1 to polyquaternium-56). A very particularly preferred conditioner is N,N-dimethyl-N-2-propenyl-2-propeneaminium chloride (polyquaternium-7).

Other examples of advantageous conditioners are cellulose derivatives and quaternized guar gum derivatives, in particular guar hydroxypropylammonium chloride (e.g. Jaguar Escel®, Jaguar C 162® (Rhodia), CAS 65497-29-2, CAS 39421-75-5). Nonionic poly-N-vinylpyrrolidone/polyvinyl acetate copolymers (e.g. Luviskol®VA 64 (BASF)), anionic acrylate copolymers (e.g. Luviflex®Soft (BASF)), and/or amphoteric amide/acrylate/methacrylate copolymers (e.g. Amphomer® (National Starch)) can also advantageously be used according to the invention as conditioners. Further examples of advantageous conditioners are quaternized silicones.

Examples of ethoxylated glyceryl mono- or di-fatty acid esters are PEG-10 olive oil glycerides, PEG-11 avocado oil glycerides, PEG-11 cocobutter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil-PEG-7 ester, PEG-6 caprylic acid/capric acid glycerides, PEG-10 olive oil glycerides, PEG-13 sunflower oil glycerides, PEG-7 hydrogenated castor oil, hydrogenated palm kernel oil glyceride PEG-6 ester, PEG-20 corn oil glycerides, PEG-18 glyceryl oleate cocoate, PEG40 hydrogenated castor oil, PEG40 castor oil, PEG-60 hydrogenated castor oil, PEG-60 corn oil glycerides, PEG-54 hydrogenated castor oil, PEG-45 palm kernel oil glycerides, PEG-80 glyceryl cocoate, PEG-60 almond oil glycerides, PEG-60 “evening primrose” glycerides, PEG-200 hydrogenated glyceryl palmate, and PEG-90 glyceryl isostearate. In the context of the present invention, PEG is polyethylene glycol and the number following PEG is the number average of the ethylene glycol units of the relevant polyethylene glycol.

Preferred ethoxylated glyceryl mono- or di-fatty acid esters are PEG-7 glyceryl cocoate, PEG-9 cocosglycerides, PEG40 hydrogenated castor oil and PEG-200 hydrogenated glyceryl palmate.

Ethoxylated glyceryl mono- or di-fatty acid esters can be used for different purposes in cosmetic preparations according to the invention. Ethoxylated glyceryl mono- or di-fatty acid esters having 3 to 12 ethylene oxide units per molecule serve as refatting agents for improving the feel of the skin after drying, and Ethoxylated glyceryl mono- or di-fatty acid esters having 30 to 50 ethylene oxide units per molecule serve as solubilizers for nonpolar substances, such as fragrances. Ethoxylated glyceryl mono- or di-fatty acid esters having more than 50 ethylene oxide units per molecule are used as thickeners.

Examples of suitable antioxidants are all antioxidants suitable or customary for cosmetic and/or dermatological applications.

Antioxidants are preferably selected from the group consisting of the amino acids (e.g. glycine, histidine, tyrosine, tryptophan), imidazoles (e.g. urocanic acid), peptides, such as D,L-carnosine, D-carnosine, L-carnosine and derivatives thereof (e.g. anserine), carotenoids, carotenes (e.g. α-carotene, β-carotene, γ-lycopene), chlorogenic acid and derivatives thereof, liponic acid and derivatives thereof (e.g. dihydroliponic acid), aurothioglucose, propylthiouracil and other thiols, (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa- and heptathionine sulfoximine) in very small doses (e.g. pmol to μmol/kg mixture according to the invention), and furthermore, metal chelators (e.g. α-hydroxy-fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA, unsaturated fatty acids (e.g. γ-linolenic acid, linoleic acid, oleic acid), folic acid, furfurylidene sorbitol, ebiquinone and ubiquinol, vitamin C and derivatives (e.g. ascorbyl palmitate, magnesium ascorbyl phosphate, ascorbyl acetate), tocopherols and derivatives (e.g. vitamin E acetate), vitamin A and derivatives (vitamin A palmitate) and coniferyl benzoate from benzoin resin, rutinic acid and derivatives thereof, α-glycosylrutin, ferulaic acid, furfurylideneglucitol, carnosine, butylhydroxytoluene, butylhydroxyanisole, nordihydroguajac resin acid, nordihydroguajaret acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, in particular trans-stilbene oxide) and suitable derivatives (salts, esters, ethers, sugars, nucleotides, nucleosides, peptides and lipids).

Suitable thickeners for cosmetic preparations according to the invention are crosslinked polyacrylic acids and derivatives thereof, carrageen, xanthan, polysaccharides, such as xanthan gum, guar guar, agar agar, alginates or tyloses, cellulose derivates, e.g. carboxymethylcellulose, hydroxycarboxymethylcellulose, hydroxyethylpropylcellulose, hydroxybutylmethylcellulose and hydroxypropylmethylcellulose, and furthermore higher molecular weight polyethylene glycol mono- and diesters of fatty acids, fatty alcohols, monoglycerides and fatty acids, polyvinyl alcohol and polyvinylpyrrolidone.

Suitable thickeners are furthermore, for example, hydrophilic pyrogenic silica gels, polyacrylamides, polyvinyl alcohol, polyvinylpyrrolidone, esters of fatty acids with polyols, such as, for example, pentaerythritol or trimethylolpropane, fatty alcohol ethoxylates having a narrow homolog distribution of C₁-C₂₀-alkyloligoglucosides and electrolytes, such as sodium chloride and ammonium chloride.

Biocides suitable for cosmetic preparations according to the invention are agents having a specific action against gram-positive bacteria, e.g. triclosan (2,4,4′-trichloro-2′-hydroxydiphenylether), chlorhexidine (1,1′-hexamethylenebis[5-(4-chlorophenyl)biguanide) and TTC (3,4,4′-trichlorocarbanilide). Suitable biocides are furthermore isothioxalones, such as, for example, 5-chloro-2-methyl-3(2H)isothiazolone and 2-methyl-3(2H)isothiazolone. Quaternary ammonium compounds are in principle also suitable and are preferably used for disinfectant soaps and wash lotions. Numerous fragrances also have biocidal properties. A large number of essential oils or characteristic ingredients thereof, such as, for example, clove oil (eugenol), mint oil (menthol), or thyme oil (thymol), also have pronounced antimicrobial activity. Further suitable biocides are fluorine compounds, which are suitable, for example, for the prevention of caries, such as, for example, NaF and amine fluorides.

Examples of foam formers may be, for example, surfactants containing sulfone groups, in particular sodium lauryl sulfate.

Examples of humectants are sorbitol, glycerol, polyethylene glycol, for example having a molecular weight Mₙ in the range of from 200 to 1000 g/mol.

Cosmetic preparations according to the invention may furthermore comprise colorants, for example dyes or pigments, glitter and/or other effect substances (e.g. color streaks).

Cosmetic preparations according to the invention may comprise, for example, one or more abrasives, for example polyethylene glycol, silica gel or calcium carbonate.

Cosmetic preparations according to the invention can be prepared, for example, by mixing a dispersion or emulsion according to the invention with one or more of the abovementioned substances from oil or fat phase resins (D) fragrances and aromas (E) and additives (F) and, if appropriate, with water.

If it is desired to use dispersions or emulsions according to the invention as release agents or in methods for separating articles they can be used, for example, for the production of peelable films or stickers or in injection molding machines for simplified demolding of injection molded parts. For example, a film-like material, e.g. a polymer film comprising polyester, polyethylene, polypropylene or polyurethane, can be brought into contact on one or two sides with dispersion or emulsion according to the invention and then dried, for example in the air. Dispersion or emulsions according to the invention is preferably mixed with a further material, for example colloidal silica, and then brought into contact with film-like material.

The invention is explained by working examples.

The K values of the terpolymers used according to the invention were determined according to H. Fikentischer, Cellulose-Chemie, volume 13, 58-64 and 761-774 (1932) in cyclohexane at 25° C. and a polymer concentration of 2% by weight.

### 1. Preparation of Terpolymer (A.1) and Terpolymer (A.2)

206 g of polyisobutene having a molecular weight Mₙ of 550 g/mol and 185 g of diisobutene were initially taken in a 4 l vessel and heated to 110° C. in a gentle stream of nitrogen. After the temperature of 110° C. had been reached, 184 g of maleic anhydride in liquid form as a melt at about 70° C. were metered in in the course of 5 hours, and 5.5 g of tert-butyl peroctanoate, dissolved in 25 g of diisobutene (mixture of 2,4,4-trimethyl-1-penten and 2,4,4-trimethyl-2-pentene), in the course of 5.5 hours. Stirring was then continued for one hour at 120° C. Terpolymer (A.1) was obtained.

The resulting reaction mixture was cooled to 90° C., and 2400 g of water and 140 g of 50% by weight aqueous sodium hydroxide solution was simultaneously added. Thereafter, stirring was effected for 4 hours at 90° C., followed by cooling to room temperature. Terpolymer (A.2) was obtained in the form of an aqueous dispersion which had a pH of 6.5 and a water content of 80% by weight. The K value was 14.7.

### 2. Preparation of Aqueous Emulsions According to the Invention

### 2.1 Preparation of Aqueous Emulsion WE-1 According to the Invention

200 g of terpolymer (A.1) in the form of a 60% by weight solution in ortho xylene and 220 g of silicone oil (linear polydimethylsiloxane, =350 mm²/s at 25° C.) was stirred in a 4 l stirred vessel and heated to 90° C. with stirring. 700 g of water and 15 mg of H(OCH₂CH₂)₃O—(CH₂)₃—Si(CH₃)[OSi(CH₃)₃]₂[OSi(CH₃)₂OSi(CH₃)₃] were added, and the ortho-xylene was then removed by steam distillation. 82 g of 25% by weight aqueous sodium hydroxide solution were then added. Aqueous emulsion WE-1 according to the invention, having a water content of 70%, was obtained.

### 2.2 Preparation of Aqueous Emulsion WE-2 According to the Invention

250 g of terpolymer (A.1) in the form of a 60% by weight solution in ortho-xylene, 91 g of polyisobutene (Mₙ=1000 g/mol) and 180 g of a silicone oil (linear polydimethylsiloxane, ν=350 mm²/s at 25° C.) were stirred in a 4 l stirred vessel and heated to 90° C. with stirring. 220 g of water and 18 mg of H(OCH₂CH₂)₃O—(CH₂)₃—Si(CH₃)[OSi(CH₃)₃]₂[OSi(CH₃)₂OSi(CH₃)₃] were added, and the ortho-xylene was then removed by steam distillation. Thereafter, 102 g of 25% by weight aqueous sodium hydroxide solution were added and dilution was effected with 1000 g of hot (90° C.) water. The mixture was then left to cool to room temperature. Aqueous emulsion WE-1 according to the invention, having a water content of 80%, was obtained.

### 2.3 Preparation of Aqueous Emulsion WE-3 According to the Invention

120 g of water were added to 250 g of terpolymer (A.2) in the form of a 30% by weight aqueous dispersion and 200 g of a silicone oil (linear polydimethylsiloxane, ν=1000 mm²/s at 25° C.) in a stirred vessel, and stirring was effected with an Ultraturrax for 2 minutes at 15 000 rpm at 25° C. Aqueous emulsion WE-3 according to the invention, having a water content of 50%, was obtained.

### 2.4 Preparation of Aqueous Emulsion WE4 According to the Invention

120 g of water were added to 250 g of terpolymer (A.2) in the form of a 30% by weight aqueous dispersion and 200 g of a silicone oil (having on average two (CH₂)₁₀—COOH groups per molecule, randomly distributed, functionalized polydimethylsiloxane, ν=1000 mm²/s at 25° C.), stirring was effected with an Ultraturrax for 2 minutes at 15 000 rpm at 25° C. Aqueous emulsion WE-4 according to the invention, having a water content of 50%, was obtained.

### 3. Use of Aqueous Emulsions According to the Invention in the Production of Leather and Comparative Examples

Preliminary remark: data in % by weight designates the amount of active substance and relates to the shaved weight, unless stated otherwise.

100 parts by weight of chrome-tanned cattle leather having a shaved thickness of from 1.8 to 2.0 mm or cut into three strips of 2000 g each and drummed over a period of 90 minutes at 30° C. in a rotatable barrel (50 l) having baffles with

200% by weight of water, 2% by weight of sodium formate, 0.4% by weight of NaHCO₃ and 2% by weight of a naphthalene sulfonic acid/formaldehyde condensate, prepared according to U.S. Pat. No. 5,186,846, example “Dispersant 1”. The liquor was then discharged. One strip each was placed together with 100% by weight of water (30° C.) in the rotatable barrels 1 to 3, and in each case 1% by weight of a dye mixture which had the following composition was added:
70 parts by weight of dye from EP-B 0 970 148, example 2.18,
30 parts by weight of Acid Brown 75 (iron complex), Color Index 1.7.16.

After a drumming time of 10 minutes (30° C.) at 10 rpm, 6% by weight of sulfone tanning agent from EP-B 0 459 168, example K1, and 2% by weight of a resin tanning agent (melamine/formaldehyde condensate) were added and drumming was effected for a further 45 minutes at 15 rpm in the barrel. Thereafter, 4% by weight of vegetable tanning agent Mimosa®, commercially available from BASF Aktiengesellschaft, and 3% by weight of the fatliquor FL-1 according to 4. (see below) were added and drumming was carried out for 45 minutes.

Thereafter a further 4% by weight of fatliquor FL-1 and, if appropriate 1% by weight of aqueous emulsion according to the invention as shown in table 1 were metered. A further 2% by weight of brown dye (see above) were then metered.

After drumming for a further 45 minutes acidification to a pH of from 3.6 to 3.8 was effected with formic acid. After a further 20 minutes, the liquor was discharged and washing was effected with 200% by weight of water. Thereafter, 100% by weight of water, 2% by weight of fatliquor FL-1 and aqueous emulsion according to the invention as shown in table 1 were metered. Acidification was effected with 1% by weight of formic acid, the liquor was discharged and washing was once again effected with water.

The washed leathers were sammed, dried and assessed according to the test criteria specified in table 1.

TABLE 1
Production of leather

| | | | | Grain | | Color | |
|---|---|---|---|---|---|---|---|
| | | Full- | Soft | tight- | | pene- | |
| Barrel | Emulsion | ness | ness | ness | Handle | tration | Levelness |
| 1 | WE-3 | 2 | 2 | 2 | “silky” | 3 | 2.5 |
| 2 | — | 2.5 | 3 | 2 | “dry” | 3 | 2.5 |
| 3 | WE-4 | 1.5 | 1.5 | 2 | “silky” | 2.5 | 1.5 |

The “silky” handle is felt to be pleasant and a “dry” handle is as a rule felt to be unpleasant.

### Remarks:

The rating was effected according to a rating system from 1 (very good) to 5 (poor).

### 4. Preparation of a Fatliquor

In a stirred vessel, 2.3 g of a polyisobutene (Mₙ: 1000 g/mol) were mixed with 300 g of n-C₁₈H₃₇O(CH₂CH₂O)₂₅H, 400 g of oleic acid and 2.3 kg of sulfited oxidized triolein and heated to 60° C. 4.7 l of water and 100 g of n-C₁₈H₃₇O(CH₂CH₂O)₇H were then added. The resulting emulsion was passed through a gap homogenizer and cooled to room temperature. Fatliquor FL-1 was obtained.

### 5. Preparation of Emulsions According to the Invention and Comparative Emulsions and Stability Investigations Thereof.

### General Method:

Terpolymer (A.2) in the form of a 30% by weight aqueous dispersion according to table 2, 100 g of a silicone oil (linear polydimethylsiloxane, ν=350 mm²/s at 25° C.), N-oleylsarcosine sodium salt according to table 2 were made up to 1 kg with water and stirred with an Ultraturrax for 2 minutes at 15 000 rpm and 25° C. Aqueous emulsion WE-5 or WE-6 according to the invention was obtained.

For the preparation of the comparative emulsion WE-7 terpolymer (A.2) was omitted and an emulsifier mixture according to table 2 was used.

The storage for the purpose of the stability investigation was effected in each case at room temperature.

TABLE 2
Comparison of the stability of emulsions according to the invention
(all data in % by weight, based on the prepared emulsion):

| | % by | | Stability after | | |
|---|---|---|---|---|---|
| | weight | | storage [days] | | |
| No. | (A.2) | Further emulsifier, amount | 7 | 14 | 55 |
| V.WE-7 | 0 | 6% by weight of N-oleoyl-N- | + | − | − |
| | | sarcosine Na salt | | | |
| | | 1% by weight of C₁₀-oxo alcohol, | | | |
| | | reacted with 7 mol of ethylene | | | |
| oxide | | | | | |
| WE-5 | 3 | 1 % of N-oleoyl-N-sarcosine Na | + | + | + |
| | | salt | | | |
| WE-6 | 5 | — | + | + | − |

+: emulsion, −: emulsion broken

1. The use of terpolymers (A), obtainable by free radical copolymerization of
(a) at least one anhydride of a C₃-C₁₀-dicarboxylic acid,
(b) at least one 1,1-di-(C₁-C₃-alkyl)-substituted C₄-C₈-olefin,
(c) polyisobutene having an average molecular weight Mₙ in the range of from 200 to 10 000 g/mol
and, optionally, hydrolysis,
for the preparation of aqueous emulsions or dispersions of silicones (B) with the use of not more than 2% by weight of further emulsifier, based on the total aqueous emulsion or dispersion.

2. A process comprising preparing an aqueous emulsion or dispersion of a silicone (B) using at least one terpolymer (A), obtainable by free radical copolymerization of
(a) at least one anhydride of a C₃-C₁₀-dicarboxylic acid,
(b) at least one 1,1-di-(C₁-C₃-alkyl)-substituted C₄-C₈-olefin,
(c) polyisobutene having an average molecular weight Mₙ in the range of from 200 to 10 000 g/mol
and, optionally, hydrolysis,
and with the use of not more than 2% by weight of further emulsifier, based on the total aqueous emulsion or dispersion.

3. The process according to claim 2, wherein the 1,1-di-(C₁-C₃-alkyl)-substituted C₄-C₈-olefin (b) is isobutene.

4. The process according to claim 2, wherein at least one anhydride of a C₃-C₁₀-dicarboxylic acid (a) is maleic anhydride.

5. The process according to claim 2, which is carried out using not more than 2% by weight of polyalkoxylated C₁₀-C₄₀-fatty alcohol as a further emulsifier, based on the total aqueous emulsion or dispersion.

6. The process according to claim 2, wherein silicone (B) is a silicone which is liquid at room temperature.

7. The process according to claim 2, wherein silicone (B) has on average at least one carboxyl group per molecule.

8. The process according to claim 2, wherein silicone (B) has no carboxyl group.

9. The process according to claim 2, wherein further comprising at least one organic solvent selected from aliphatic and aromatic hydrocarbons which are liquid at room temperature are mixed and organic solvent is then separated off.

10. A dispersion or emulsion obtainable by a process according to claim 2.

11. A method comprising producing fibrous substrates, as release agents, as cleaning agents, as lubricants, for the treatment or processing of construction materials or in cosmetic preparations utilizing the dispersion or emulsion according to claim 10.

12. The method according to claim 11, wherein fibrous substrates are selected from leather, paper, wood, textile and board.

13. A process for the production of fibrous substrates, for the cleaning of surfaces, for the separation of articles, for the treatment or processing of construction materials or of cosmetic preparations utilizing the dispersion or emulsion according to claim 10.

14. The process according to claim 13, wherein fibrous substrates are selected from leather, paper, wood, textile and board.

15. A method for reducing the friction between moving parts using dispersions or emulsions according to claim 10.

16. A leather, cosmetic preparation or construction material produced using dispersions or emulsions according to claim 10.

17. The process according to claim 3, wherein at least one anhydride of a C₃-C₁₀-dicarboxylic acid (a) is maleic anhydride.

18. The process according to claim 3, which is carried out using not more than 2% by weight of polyalkoxylated C₁₀-C₄₀-fatty alcohol as a further emulsifier, based on the total aqueous emulsion or dispersion.

19. The process according to claim 4, which is carried out using not more than 2% by weight of polyalkoxylated C₁₀-C₄₀-fatty alcohol as a further emulsifier, based on the total aqueous emulsion or dispersion.

20. The process according to claim 3, wherein silicone (B) is a silicone which is liquid at room temperature.
